(19) United States
(12) Patent Application Publication
(10) Pub. No.: US 2009/0198122 A1
(43) Pub. Date: Aug. 6, 2009

(54) SYSTEMS AND METHODS FOR DETERMINING METABOLIC RATE USING TEMPERATURE SENSITIVE MAGNETIC RESONANCE IMAGING

(76) Inventors: John Pile-Spellman, Pelham, NY (US); Erwin Lin, Whitestone, NY (US)

Correspondence Address: KENYON & KENYON LLP, ONE BROADWAY, NEW YORK, NY, 10004, US

(21) Appl. No.: 12/161,520

(22) PCT Filed: Jan. 22, 2007
(86) PCT No.: PCT/US07/01797

§ 371 Date: Nov. 18, 2008

(30) Foreign Application Priority Data
Jan. 25, 2006 (US) 60/761772

(51) Int. Cl. A61B 5/055

(52) U.S. Cl. 600/412; 600/419

(57) ABSTRACT

A method for determining a metabolic rate of a portion of a body of a patient. The method includes obtaining magnetic resonance information from the portion of the body after introduction of a fluid and determining a magnetic resonance parameter using the magnetic resonance information. The method further includes using the magnetic resonance parameter to determine a temperature differential in the portion of the body and using the temperature differential to determine a metabolic rate of the portion of the body.

### CROSS-REFERENCE TO RELATED APPLICATIONS

The present application claims the benefit of and priority to International Patent Application No. PCT/US07/01797, filed 22 Jan. 2007, which claims the benefit of and priority to U.S. Provisional Patent Application No. 60/761,772, filed 25 Jan. 2006, both of which are expressly incorporated herein in their entireties by reference thereto.

The present application is related to co-pending applications “Systems and Methods for Determining a Cardiovascular Parameter Using Temperature Sensitive Magnetic Resonance Imaging,” filed herewith and “Systems and Methods for Imaging a Blood Vessel Using Temperature Sensitive Magnetic Resonance Imaging,” filed herewith. Both of these applications are incorporated by reference herein.

### FIELD OF THE INVENTION

The present invention relates to systems and methods for calculating metabolic rate based on a temperature differential determined from information obtained by magnetic resonance imaging.

### BACKGROUND

Metabolic rate is the rate at which heat is emitted by the entire body of a person at rest or during activity. In addition, metabolic rate of a portion of the body, such as an individual organ like the brain, is the rate at which heat is emitted by the portion of the body. Methods of calculating metabolic rate of the entire body often involve continuous measurements of heat output (direct calorimetry) or exhaled gas exchange (indirect calorimetry) in people confined to metabolic chambers. A metabolic chamber is a small room a person can live in for a 24 hour period, while metabolic rate is measured during meals, sleep, and light activities. The heat released from a person's body is measured to determine how much energy each activity has burned for that person. Using indirect calorimetry, oxygen consumption, carbon dioxide production and nitrogen excretion are measured to calculate a ratio that reflects energy expenditure.

Non-invasive methods of calculating relative metabolic rate based on oxygen (or glucose) consumption have been performed in animals using positron emission computed tomography (“PET”) and magnetic resonance imaging (“MRI”). Animal experiments using blood oxygen level dependent (“BOLD”) MRI have been utilized to allow indirect assessment of oxygen levels in blood and calculate relative metabolic rates of oxygen consumption in body organs such as the brain. Animal experiments have also been performed using ¹⁷O and ¹³C MRI spectroscopy to estimate relative metabolic rate based on oxygen and glucose metabolism. These methods, however, only provide indirect relative measures of metabolic rate and they are not readily implemented in humans.

A need therefore exists for a MRI method and system for measuring the temperature differential and blood flow rate at the arterial input and venous output of a portion of the body in order to determine metabolic rate.

### SUMMARY OF THE INVENTION

Systems and methods for determining metabolic rate using temperature sensitive magnetic resonance imaging are provided. In an embodiment, the present invention provides a method for determining a metabolic rate of a portion of a body of a patient. The method comprises introducing a fluid into a blood vessel of the patient and obtaining magnetic resonance information from the portion of the body. The method further comprises determining a magnetic resonance parameter from the portion of the body using the magnetic resonance information and determining a temperature differential in the portion of the body using the magnetic resonance parameter. The method further comprises determining the metabolic rate using the temperature differential.

In an embodiment, the present invention provides a machine-readable medium having stored thereon a plurality of executable instructions, which, when executed by a processor, performs obtaining magnetic resonance information from a portion of a body of a patient after introduction of fluid into a blood vessel of the patient and determining a magnetic resonance parameter from the portion of the body using the magnetic resonance information. The plurality of executable instructions further performs determining a temperature differential in the portion of the body using the magnetic resonance parameter and determining a metabolic rate of the portion of the body using the temperature differential. The plurality of executable instructions further performs determining the metabolic rate using the temperature differential.

### BRIEF DESCRIPTION OF THE DRAWINGS

FIG. 1 is a flow diagram that illustrates an embodiment of a method of calculating metabolic rate using temperature sensitive MRI.

FIG. 2 illustrates an embodiment of a system to control the temperature and flow of fluid introduced into a patient.

FIG. 3 is a block diagram that depicts an embodiment of a user computing device.

FIG. 4 is a block diagram that depicts an embodiment of a network architecture.

### DETAILED DESCRIPTION OF THE INVENTION

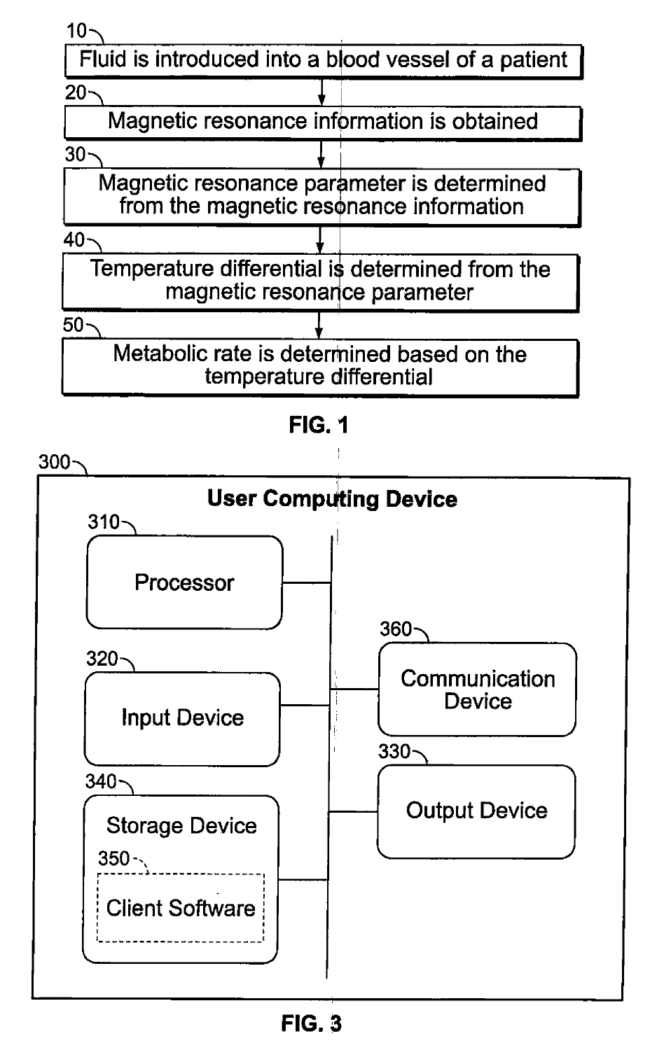

In an embodiment, the present invention provides a method for determining a metabolic rate of a portion of a body of a patient based on a temperature differential in the portion of the body determined from information obtained by MRI. Specifically, referring to FIG. 1, a method for determining a metabolic rate comprises introducing a fluid into a blood vessel of a patient (10) and obtaining magnetic resonance information from the portion of the body of the patient (20). A magnetic resonance parameter is determined using the magnetic resonance information (30) and a temperature differential in the portion of the body is determined using the magnetic resonance parameter (40). Based on the temperature differential, a metabolic rate is determined (50).

The metabolic rate can be for a portion of the body, such as an organ or tissue. Non-limiting examples of organs for which a metabolic rate can be determined include the brain, lungs, heart, kidney, liver, stomach and other gastrointestinal organs, and vasculature. Vasculature includes arteries and veins including central and peripheral arteries and veins. For example, the artery can be the carotid artery and the vein can be an internal jugular vein or a large vein draining an organ.

Referring again to FIG. 1, with respect to introducing a fluid into a blood vessel of a patient (10), the fluid can be any biologically compatible fluid that can perfuse the portion of the body. For example, the fluid may be water, blood or a saline solution. The fluid can be introduced over any time frame at any rate sufficient to induce temperature changes that can be effectively imaged. For example, the fluid may be introduced at a constant rate over a period of seconds, such as, for example, a bolus injection where the shape of the input is a square wave. Alternatively, the fluid may be introduced over a period of minutes, where the shape of the input is a desired function of time including a sinusoidal function. Furthermore, the shape of the input may be designed to optimize the arterial input function of the blood vessel being imaged and thereby simplify calculations.

The fluid can be introduced in any manner such that the fluid can perfuse the portion of the body and induce temperature changes that can be effectively imaged. For example, the fluid can be injected intravenously or intra-arterially or introduced as a gas into the lungs via inhalation. Further, the fluid can be introduced at a site local or distant to the portion of the body in which the metabolic rate is being determined. For example, the fluid may be injected into a peripheral vein using a conventional intravenous line, into a central vein using a central venous line or through a catheter in a central or peripheral artery that supplies the portion of the body in which metabolic rate is being calculated. The temperature of the introduced fluid can be above or below body temperature. Further, the temperature of the introduced fluid may have a uniform constant temperature below or above body temperature or can vary over time and include temperatures above and below body temperature. For example, the introduced fluid may vary over time when the injection site is remote from the tissue of interest, such as a peripheral vein, and the profile of the injected fluid changes after passing through the heart and pulmonary circulation. Using an injection with a time-varying temperature may reduce such changes. A constant temperature injection may be used, for example, when the injection site is closer to the tissue of interest, such as a central artery, and the profile of the injected fluid does not change as readily.

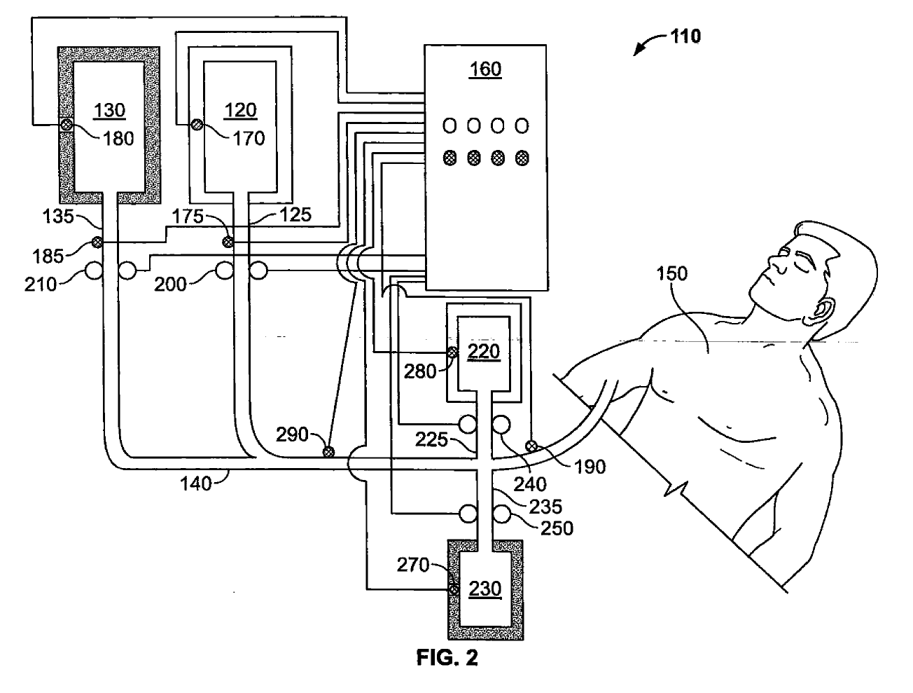

A system can be used for controlling the temperature of the fluid that is introduced into the patient by combining fluids having two different temperatures and introducing the combined fluid into the patient. Referring to FIG. 2, in an embodiment, such a system 110 includes first reservoir 120 containing a first fluid at a temperature below body temperature and second reservoir 130 containing a second fluid at a temperature above body temperature. First and second reservoirs 120 and 130 are in fluid communication with respective first and second fluid lines 125 and 135, which, in turn, are in fluid communication with a convergent line 140. First and second lines 125 and 135 can converge with convergent line 140 via a Y-connector, for example, such that the fluid outflow of reservoirs 120 and 130 is combined into a single fluid line. System 110 further comprises third reservoir 220 containing a third fluid at a temperature below body temperature and fourth reservoir 230 containing a fourth fluid at a temperature above body temperature. Third and fourth reservoirs 220 and 230 are in fluid communication with respective third and fourth fluid lines 225 and 235, which, in turn, are in fluid communication with convergent line 140. Convergent line 140 is insertable into a blood vessel of a patient 150 either directly or indirectly, via a catheter attached to the distal end of convergent line 140.

System 110 further comprises first reservoir temperature sensor 170 in communication with first reservoir 120 and first line temperature sensor 175 in communication with first fluid line 125. System 110 further comprises second reservoir temperature sensor 180 in communication with second reservoir 130 and second line temperature sensor 185 in communication with second fluid line 135. System 110 further comprises third reservoir temperature sensor 280 in communication with third reservoir 220 and fourth reservoir temperature sensor 270 in communication with fourth reservoir 230. In addition, system 110 comprises convergent line temperature sensor 190 and 290. System 110 further comprises controller 160 for controlling the flow of first, second, third and fourth fluids from respective first, second, third and fourth reservoirs 120, 130, 220, and 230. Specifically, in an embodiment, controller 160 is in communication with sensors 170, 180, 175, 185, 190, 270, 280 and 290. Controller 160 is also in communication with first pump 200, second pump 210, third pump 240 and fourth pump 250 which, in turn, are in communication with first fluid line 125, second fluid line 135, third fluid line 225 and fourth fluid line 235 respectively. A non-limiting example of first, second, third and fourth pumps 200, 210, 240 and 250 are power injectors. In certain embodiments, a system does not include third and fourth pumps. In order to control the flow of first and second fluids, controller 160 receives temperature input signals from sensors 170, 180, 175, and 185 regarding the temperature of the first and second fluids and accordingly sends out a control signal to pumps 200 and 210 to adjust the flow rate of the fluids. Likewise, in order to control the flow of third and fourth fluids, controller 160 receives temperature input signals from sensors 280 and 270 regarding the temperature of the third and fourth fluids and accordingly sends out a control signal to pumps 240 and 250 to adjust the flow rate of the fluids. Controller 160 may be computerized and the flow rate of first and second fluids exiting respective first and second reservoirs 120 and 130 can be varied in accordance with a look-up table or an algorithm to achieve a desired temperature variation of the introduced combined fluid. Temperature readings from the convergent line temperature sensors 190 and 290 can be used to confirm the expected temperature in convergent line 140 as determined from the look-up table or the algorithm. Controller 160 may be computerized and may introduce additional fluid from third and fourth reservoirs 220 and 230 in accordance with a look-up table or an algorithm to make adjustments to achieve the desired temperature variation of the introduced fluid or to optimize or adjust the leading and trailing edges of the introduced fluid. In one variation of the algorithm used to achieve a desired temperature variation of the fluid, repetitive injections of the fluid can be made and the algorithm adjusted accordingly.

Referring back to FIG. 1, an embodiment of a method of the present invention includes obtaining magnetic resonance information from the portion of the body in which metabolic rate is being determined (20). Specifically, magnetic resonance information is obtained from the arterial input and the venous output of the portion of the body. Such arterial input and venous output can be the blood supply and drainage of the portion of the body. The magnetic resonance information is determined by physical properties of the portion of the body and includes but is not limited to MR signal intensity, phase information, frequency information and any combination thereof. To obtain such magnetic resonance information, the patient is placed in a MR scanner and radiofrequency (RF) pulses are transmitted to the patient. The RF pulse sequences can be used to excite a slice, a series of slices or a volume containing the portion of the body. RF pulses can be applied in a dynamic fashion so that magnetic resonance information is measured dynamically, such as at multiple sequential points in time. For example, magnetic resonance information can be measured before, during and after the introduced fluid perfuses the portion of the body of the patient. The pulse sequences may include but are not limited to echo-planar, gradient echo, spoiled gradient echo and spin echo. For each slice, series of slices or volume, the magnetic resonance information can be spatially encoded by using magnetic field gradients including phase-encoding gradients and frequency-encoding gradients. Specifically, spatial encoding of the magnetic resonance information can be achieved by applying additional magnetic field gradients after excitation of tissue but before measurement of the magnetic resonance information (phase-encoding gradient) as well as during signal measurement (frequency-encoding gradient). In order to fully spatially encode a slice or volume of excited tissue, the excitation and measurement process can be repeated multiple times with different phase-encoding gradients. When performing a volume acquisition, two different phase encoding gradients can be applied in order to ultimately divide the volume into multiple slices. Spatial encoding allows calculation of the amount of magnetic resonance information emitted by small volume elements (voxels) in the excited slice or volume and therefore allows magnetic resonance information to be measured on a voxel-by-voxel basis in each slice, series of slices or volume.

The magnetic resonance information obtained in 20 is used to determine a magnetic resonance parameter in the portion of the body (30) according to an embodiment of a method of the present invention. The magnetic resonance parameter is determined by the physical properties of the portion of the body and non-limiting examples of magnetic resonance parameters includes phase changes resulting from changes in water proton resonance frequency; changes in T1 relaxation time; changes in diffusion coefficients; phase changes as determined by analysis of spectroscopic data; and any combination thereof. Methods for calculating such magnetic resonance parameters involve using well-known mathematical formulas based on the pulse sequence used and the specific parameter that is to be calculated. Methods of the present invention include measuring a single magnetic resonance parameter or multiple magnetic resonance parameters. The magnetic resonance parameter can be calculated on a voxel-by-voxel basis for each slice, series of slices or volume.

The magnetic resonance parameter determined in 30 is used to determine a temperature differential in the portion of the body (40) according to an embodiment of a method of the present invention. Specifically, a temperature differential in the arterial input and the venous output of the portion of the body is determined using the magnetic resonance parameter. Methods for calculating a temperature differential based on the above-identified magnetic resonance parameters are well-known in the art. For example, if the magnetic resonance parameter is phase changes corresponding to changes in water proton resonance frequency, a corresponding temperature differential can be calculated in accordance with the equation ΔT=ΔΦ(T)/αγTEB₀, where α is a temperature dependent water chemical shift in ppm per C⁰, γ is the gyromagnetic ratio of hydrogen, TE is the echo time; B₀ is the strength of the main magnetic field; and ΔΦ is phase change. With respect to calculating a temperature differential based on changes in T1 relaxation time, changes in diffusion coefficients, or phase changes as determined by analysis of spectroscopic data, such calculations can be performed, for example, in accordance with the methods described by Quesson and Kuroda (e.g. B Quesson, J A de Zwart & C T W Moonen. “Magnetic Resonance Temperature Imaging for Guidance of Thermotherapy;” 12 J Mag Res Img 525 (2000); K Kuroda, R V Mulkern, K Oshio et al. “Temperature Mapping using the Water Proton Chemical Shift; Self-referenced Method with Echo-planar Spectroscopic Imaging,” 43 Magn Reson Med 220 (2000), both of which are incorporated by reference herein.) Of course, as one skilled in the art will appreciate, other methods could also be employed. Notwithstanding which magnetic resonance parameter is used to calculate a temperature differential, the measured temperature change in a voxel will correspond to the concentration of indicator (for example, heat or cold) within the voxel over time.

The temperature differential determined in 40 is used to calculate the metabolic rate of the portion of the body (50). Specifically, the temperature differential is used to calculate the difference in heat flow through the arterial input of the portion of the body and the venous output of the portion of the body. The quantity of heat (H) in a volume of tissue (V) at temperature T is calculated according to the formula H=T×(V)×(specific heat)×(specific gravity). Likewise, a temperature differential (ΔT) of a volume of tissue (V) corresponds to a change in the quantity of heat (ΔH) in V according to the formula ΔH=(ΔT)×(V)×(specific heat)×(specific gravity). As a portion of the body of a patient produces heat, there is near instantaneous transfer of the heat to the blood perfusing the portion of the body. Furthermore, the total arterial blood flow (F) into a portion of the body must necessarily equal the total venous blood flow out of the portion of the body. Therefore, if the temperature differential (ΔT) between the arterial input and the venous output of the portion of the body is measured, the metabolic rate (M) of the portion of the body, can be calculated according to the formula M=(ΔT)×F×(specific heat)×(specific gravity).

For example, if a known quantity of heat, Q, is injected into the arterial input, such as, for example, using a central arterial catheter, the temperature differential in the arterial input downstream from the injection site can be measured as a function of time and the blood flow, F, can be calculated according to the equation F=Q/∫₀^{∞}H(t)dt, where H=(ΔT)×(V)×(specific heat)×(specific gravity). If the portion of the body is the brain, for example, and the total blood flow (F) in both internal carotid arteries is determined, the metabolic rate of the brain (M) can be calculated according to the formula M=(ΔT)×F×(specific heat)×(specific gravity), where (ΔT) is the temperature differential between the internal carotid arteries and the jugular veins.

In another embodiment, the present invention provides a machine-readable medium having stored thereon a plurality of executable instructions, which, when executed by a processor, performs obtaining magnetic resonance information from a portion of a body of a patient after introduction of fluid into a blood vessel of the patient. The plurality of executable instructions further performs determining a magnetic resonance parameter in the portion of the body using the magnetic resonance information and determining a temperature differential in the portion of the body using the magnetic resonance parameter. The plurality of executable instructions further performs determines the metabolic rate using the temperature differential

Referring to FIG. 3, the above-mentioned method may be performed by a user computing device 300 such as a MRI machine, workstation, personal computer, handheld personal digital assistant (“PDA”), or any other type of microprocessor-based device. User computing device 300 may include a processor 310, input device 320, output device 330, storage device 340, client software 350, and communication device 360. Input device 320 may include a keyboard, mouse, pen-operated touch screen, voice-recognition device, or any other device that accepts input. Output device 330 may include a monitor, printer, disk drive, speakers, or any other device that provides output. Storage device 340 may include volatile and nonvolatile data storage, including one or more electrical, magnetic or optical memories such as a RAM, cache, hard drive, CD-ROM drive, tape drive or removable storage disk. Communication device 360 may include a modem, network interface card, or any other device capable of transmitting and receiving signals over a network. The components of user computing device 300 may be connected via an electrical bus or wirelessly. Client software 350 may be stored in storage device 340 and executed by processor 310, and may include, for example, imaging and analysis software that embodies the functionality of the present invention

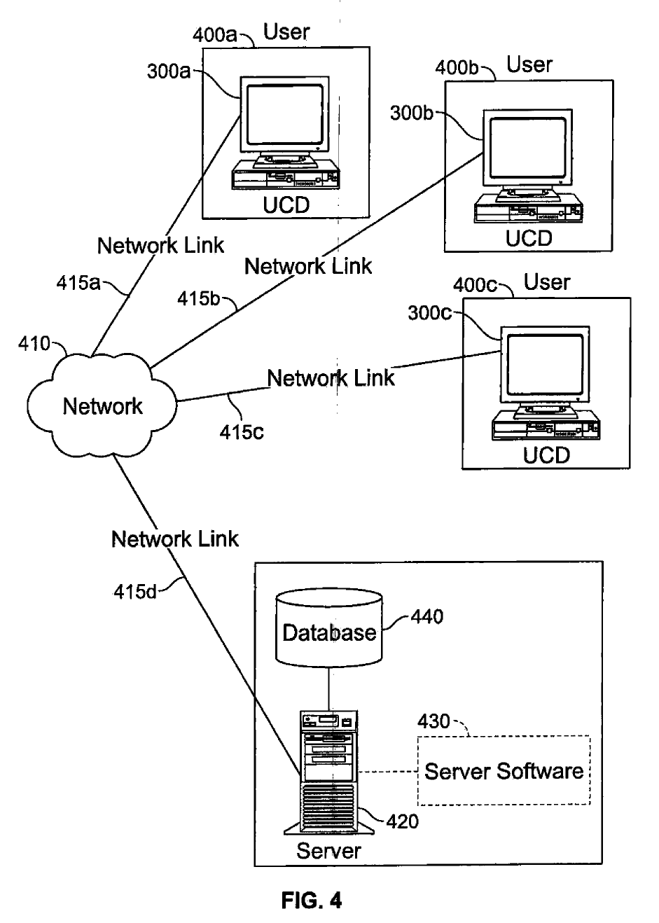

Referring to FIG. 4, the analysis functionality may be implemented on more than one user computing device 300 via a network architecture. For example, user computing device 300 may be an MRI machine that performs the obtaining of magnetic resonance information and determination functionalities. In another embodiment, user computing device 300a may be a MRI machine that performs the obtaining of magnetic resonance information functionality and the magnetic resonance parameter determination functionality, and then transfers this determination over network 410 to server 420 or user computing device 300b or 300c for all other determination functionalities.

Referring again to FIG. 4, network link 415 may include telephone lines, DSL, cable networks, T1 or T3 lines, wireless network connections, or any other arrangement that implements the transmission and reception of network signals. Network 410 may include any type of interconnected communication system, and may implement any communications protocol, which may be secured by any security protocol. Server 420 includes a processor and memory for executing program instructions, as well as a network interface, and may include a collection of servers. Server 420 may include a combination of servers such as an application server and a database server. Database 440 may represent a relational or object database, and may be accessed via server 420.

User computing device 300 and server 420 may implement any operating system, such as Windows or UNIX. Client software 350 and server software 430 may be written in any programming language, such as ABAP, C, C++, Java or Visual Basic.

The foregoing description has been set forth merely to illustrate the invention and are not intended as being limiting. Each of the disclosed aspects and embodiments of the present invention may be considered individually or in combination with other aspects, embodiments, and variations of the invention. In addition, unless otherwise specified, none of the steps of the methods of the present invention are confined to any particular order of performance. Modifications of the disclosed embodiments incorporating the spirit and substance of the invention may occur to persons skilled in the art and such modifications are within the scope of the present invention. Furthermore, all references cited herein are incorporated by reference in their entirety.

What is claimed is:

1. A method for determining a metabolic rate of a portion of a body of a patient comprising:
introducing a fluid into a blood vessel of a patient;
obtaining magnetic resonance information from the portion of the body;
determining a magnetic resonance parameter from the portion of the body using the magnetic resonance information;
determining a temperature differential in the portion of the body using the magnetic resonance parameter; and
determining a metabolic rate in the portion of the body using the temperature differential.

2. The method of claim 1, wherein determining a metabolic rate comprises determining a heat flow in the portion of the body using the temperature differential and determining a metabolic rate using the heat flow.

3. The method of claim 2, wherein determining a heat flow comprises using a heat content and a blood flow of the portion of the body, the heat content and blood flow calculated using the temperature differential in the portion of the body.

4. The method of claim 1, wherein the portion of the body is an organ.

5. The method of claim 4, wherein the organ is a brain.

6. The method of claim 1, wherein the portion of the body is an artery or a vein.

7. The method of claim 1, wherein the temperature of the introduced fluid is below body temperature of the patient.

8. The method of claim 1, wherein obtaining the magnetic resonance information comprises:
placing the patient in a magnetic resonance scanner;
transmitting radiofrequency pulses to the patient to excite a slice, a series of slices or a volume containing the portion of the body; and
measuring the magnetic resonance information from the portion of the body.

9. The method of claim 1, wherein obtaining magnetic resonance information comprises collecting the magnetic resonance information at multiple sequential points in time from the portion of the body.

10. The method of claim 9, wherein collecting the magnetic resonance information at multiple sequential points comprises collecting the magnetic resonance information before, during and after the introduced fluid perfuses the portion of the body of the patient.

11. The method of claim 1, wherein determining the magnetic resonance information comprises measuring the magnetic resonance information on a slice-by-slice or volume basis through the portion of the body of the patient.

12. The method of claim 1, wherein the determining the magnetic resonance parameter comprises determining the magnetic resonance parameter on a voxel-by-voxel basis through the portion of the body of the patient.

13. The method of claim 1, wherein the magnetic resonance parameter comprises changes in water proton resonance frequency and the temperature differential is determined using the changes in water proton resonance frequency.

14. The method of claim 1, wherein the magnetic resonance parameter comprises changes in T1 relaxation time of water protons and the temperature differential is determined using the changes in T1 relaxation time.

15. The method of claim 1, wherein the magnetic resonance parameter comprises changes in a diffusion coefficient of water in the portion of the body and the temperature differential is determined using the changes in the diffusion coefficient.

16. The method of claim 1, wherein the magnetic resonance parameter comprises changes in magnetic resonance spectroscopy measurements of the portion of the body and the temperature differential is determined using the changes in magnetic resonance spectroscopy measurements.

17. A method for determining a metabolic rate of a portion of a body of a patient comprising:
introducing a gas into a lung of the patient;
obtaining magnetic resonance information from the portion of the body;
determining a magnetic resonance parameter from the portion of the body using the magnetic resonance information;
determining a temperature differential in the portion of the body using the magnetic resonance parameter; and
determining a metabolic rate in the portion of the body using the temperature differential.

18. A machine-readable medium having stored thereon a plurality of executable instructions, which, when executed by a processor, perform the following:
obtaining magnetic resonance information from a portion of a body of a patient after introduction of a fluid into a blood vessel of the patient;
determining a magnetic resonance parameter in the portion of the body using the magnetic resonance information;
determining a temperature differential in the portion of the body using the magnetic resonance parameter; and
determining a metabolic rate of the portion of the body using the temperature differential.

19. The machine-readable medium of claim 18, wherein determining a magnetic resonance parameter in the portion of the body comprises measuring the magnetic resonance information on a voxel-by-voxel basis.

20. The machine-readable medium of claim 18, wherein obtaining the magnetic resonance information comprises obtaining the magnetic resonance information before, during and after blood perfuses the portion of the body.

21. A system for determining a metabolic rate of a portion of a body of a patient comprising:
means for introducing a fluid into a blood vessel of the patient;
means for obtaining magnetic resonance information from the portion of the body;
means for determining a magnetic resonance parameter from the portion of the body using the magnetic resonance information;
means for determining a temperature differential in the portion of the body using the magnetic resonance parameter; and
means for determining the metabolic rate using the temperature differential.

22. The system of claim 21, wherein the means for introducing a fluid comprises a central arterial catheter.

23. The system of claim 21, wherein the means for introducing a fluid comprises a central venous catheter.

24. The system of claim 21, wherein the means for introducing a fluid comprises a peripheral venous catheter.

25. The system of claim 21, wherein the means for determining a temperature differential comprises means for calculating changes in water proton resonance frequency and using the changes in water proton resonance frequency to determine the temperature differential
